(19) United States
(12) Patent Application Publication
(10) Pub. No.: US 2019/0388074 A1
(43) Pub. Date: Dec. 26, 2019

(54) TISSUE REMOVAL DEVICES AND RELATED METHODS

(71) Applicant: Boston Scientific Scimed, Inc., Maple Grove, MN (US)

(72) Inventors: Richard C. TAH, Milford, MA (US); Ashley TAYLOR, Harrisburg, PA (US); Brian P. WATSCHKE, Minneapolis, MN (US); Robert Edward LUND, Saint Michael, MN (US); Niraj Prasad RAUNIYAR, Plymouth, MN (US); Michael S. H. CHU, Brookline, MA (US)

(73) Assignee: Boston Scientific Scimed, Inc., Maple Grove, MN (US)

(21) Appl. No.: 16/447,212

(22) Filed: Jun. 20, 2019

Related U.S. Application Data
(60) Provisional application No. 62/688,784, filed on Jun. 22, 2018.

(51) Int. Cl. A61B 10/06; A61B 10/04; A61B 17/29; A61B 17/295

(52) U.S. Cl. CPC A61B 10/06; A61B 10/04; A61B 2017/00353; A61B 17/295; A61B 2017/2902; A61B 17/2909

(57) ABSTRACT

A tissue removal device may include a handle. The device may include a sheath coupled to the handle. A first portion of the sheath may be within the handle. A second portion of the sheath may protrude from the handle. The sheath may include a passage. The device may include an end effector coupled to an end of the sheath. The device may include a drive member coupled to the handle and to the end effector. The drive member may extend through the passage of the sheath to the end effector. Actuation of the handle may move the end effector between an open state, for receiving material, and a closed state, for holding onto material, by causing relative movement between the drive member and the sheath. Further actuation of the handle may move the end effector relative to the handle while the end effector remains in the closed state.

### CROSS-REFERENCE TO RELATED APPLICATIONS

This patent application claims the benefit of priority under 35 U.S.C. § 119 to U.S. Provisional Patent Application No. 62/688,784, filed Jun. 22, 2018, which is herein incorporated by reference in its entirety.

### TECHNICAL FIELD

Aspects of the present disclosure relate to tissue removal devices and related methods.

### BACKGROUND

A tissue removal device may be used to obtain a piece of tissue from a subject. One example of a tissue removal device is an endoscopic biopsy device, which may be used to obtain a piece of tissue for microscopic examination to determine malignancy, while subjecting the subject to a minimal amount of trauma. Typically, the device may include a long and thin flexible shaft that may extend from a proximal handle to a distal end effector. In some instances, the end effector may include forceps having a pair of opposing jaws. The jaws may be manipulated via the handle to cause the jaws to open and close on a tissue mass from which a sample is needed. The jaws may close around a small piece of the tissue mass. The small piece may be captured between the jaws. A tug or pull on the device may sever the captured tissue from the rest of the tissue mass. The device then may be withdrawn, and the piece of tissue may be removed from the jaws for analysis.

It is sometimes difficult for the jaws to tightly grasp tissue at a sampling site so as to harvest the tissue sample for analysis. This can be due to the nature of the tissue involved, such as cutting through the tough muscle tissue of the bladder, as well as the nature of the jaws which may slip without biting deep enough into the tissue mass. It can also result from the operator not fully closing the jaws down hard enough from actuation of the handle. Accordingly, a tug on the device to harvest the sample may result in the jaws slipping away from the tissue mass. Moreover, the jaws may cut close to a blood vessel, leading to bleeding. The tissue removal devices and related methods of the present disclosure address at least some of the aforementioned issues.

### SUMMARY

Aspects of the present disclosure relate to tissue removal devices and related methods. Each of the aspects disclosed herein may include one or more of the features described in connection with any of the other disclosed aspects.

In one aspect of the present disclosure, a tissue removal device may include a handle assembly. The tissue removal device also may include a sheath coupled to the handle assembly. A first portion of the sheath may be within the handle assembly. A second portion of the sheath may protrude from the handle assembly. The sheath may include a passage extending therethrough. The tissue removal device also may include an end effector coupled to an end of the sheath. The tissue removal device may further include a drive member coupled to the handle assembly and to the end effector. The drive member may extend through the passage of the sheath to the end effector. Actuation of the handle assembly may move the end effector between an open state, for receiving material, and a closed state, for holding onto material, by causing relative movement between the drive member and the sheath. Further actuation of the handle assembly may move the end effector relative to the handle assembly while the end effector remains in the closed state.

Aspects of the tissue removal device may include one or more of the features below. The handle assembly may include a handle body and a handle actuator. The handle actuator may be movable relative to the handle body. Actuation of the handle assembly may include moving the handle actuator relative to the handle body. The further actuation of the handle assembly may draw the end effector toward the handle body while the end effector remains in the closed state. The further actuation of the handle assembly may draw the sheath toward the handle body while the end effector remains in the closed state. The handle assembly may include a first member movably mounted on the handle body. The first member may be fixedly coupled to the drive member. The handle assembly also may include a second member movably mounted on the handle body. The second member may be fixedly coupled to the sheath. The first member may be movably coupled to the second member. The second member may include one or more latching elements. In a first state of the one or more latching elements, the one or more latching elements may latch the second member to the handle body, such that the second member may be positionally fixed on the handle body. In a second state of the one or more latching elements, the one or more latching elements may release the second member from the handle body, such that the second member may be movable relative to the handle body. Movement of the first member may move the one or more latching elements to the first state. Movement of the first member may move the one or more latching elements to the second state. A biasing member may have a first end engaging the first member and a second end engaging the second member. The biasing member may be configured to bias at least one of the first member and the second member toward a configuration in which the first member is a predetermined distance from the second member, to move the end effector toward the closed state. The further actuation of the handle assembly may move the end effector relative to the handle assembly by translationally moving the end effector along an axis coaxial with a longitudinal axis of the sheath. The further actuation of the handle assembly may move the end effector relative to the handle assembly by rotating the end effector about an axis coaxial with a longitudinal axis of the sheath.

In another aspect of the present disclosure, a tissue removal device may include a handle assembly supporting a first member and a second member. The tissue removal device also may include a sheath. A proximal portion of the sheath may extend into the handle assembly. A distal portion of the sheath may protrude from the handle assembly. The sheath may include a passage extending therethrough. A proximal end of the sheath may be fixedly coupled to the first member. The tissue removal device also may include an end effector coupled to a distal end of the sheath. The tissue removal device may further include a drive member. The drive member may extend through the passage of the sheath. A distal end of the drive member may be coupled to the end effector. A proximal end of the drive member may be fixedly coupled to the second member. The first member and the second member may be coupled so as to be movable relative to each other in a first state of the tissue removal device, and to move together as a unit in a second state of the tissue removal device.

Aspects of the tissue removal device may include one or more of the features below. The first member and the second member may be coupled by one or more pins. The second member may be movable away from the first member by sliding along the one or more pins when the tissue removal device is in the first state. The second member may be prevented from moving away from the first member by the one or more pins when the tissue removal device is in the second state. A compression spring may engage opposing surfaces of the first member and the second member. The first member may include one or more latching elements. The one or more latching elements may be movable between a latching state, in which the one or more latching elements latch the first member to the handle assembly, thereby positionally fixing the first member relative to the handle assembly, and a releasing state, in which the one or more latching elements unlatch from the handle assembly, thereby allowing the first member to move relative to the handle assembly. Movement of the second member relative to the first member may cause the second member to move the one or more latching elements between the latching state and the releasing state.

In another aspect of the present disclosure, a tissue removal method that uses a tissue removal device to remove a tissue specimen from a tissue mass of a subject, may include holding a handle assembly of the tissue removal device. The method also may include guiding a shaft of the tissue removal device and an end effector of the tissue removal device toward the tissue mass to position the end effector proximate a target area of the tissue mass. The shaft may protrude from the handle assembly. The shaft may include a sheath having an end supporting the end effector. The shaft may further include a drive member extending through the sheath, the drive member being coupled to the end effector. The method also may include actuating the handle assembly to move the end effector between an open state for receiving a tissue specimen from the target area and a closed state for holding onto the target area. Movement of the end effector between states may be at least partially driven by movement of the drive member relative to the sheath. The method also may include further actuating the handle assembly to move the end effector relative to the handle assembly while the end effector remains in the closed state to pull the target area away from the rest of the tissue mass.

Aspects of the tissue removal method may include one or more of the features below. At least one of actuating the handle assembly and further actuating the handle assembly may include rotating the end effector about an axis coaxial with a longitudinal axis of the sheath. Further actuating the handle assembly to move the end effector relative to the handle assembly may include translationally moving the end effector along an axis coaxial with a longitudinal axis of the sheath.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate aspects of the present disclosure and together with the description, serve to explain the principles of the disclosure.

FIG. 1 shows a partially transparent view of a tissue removal device, in accordance with aspects of the present disclosure.

FIG. 2 shows a cross-sectional view of the tissue removal device of FIG. 1, in accordance with aspects of the present disclosure.

FIG. 3 shows a close-up view of an area of the tissue removal device of FIG. 1, in accordance with aspects of the present disclosure.

FIG. 4 shows a close-up view of another area of the tissue removal device of FIG. 1, in accordance with aspects of the present disclosure.

FIG. 5 shows a close-up perspective view of a proximal end portion of a drive member of the tissue removal device of FIG. 1, in accordance with aspects of the present disclosure.

FIG. 6 shows a perspective view of a drive assembly of the tissue removal device of FIG. 1, in accordance with aspects of the present disclosure.

FIG. 7 shows a perspective view of a holder of the drive assembly of FIG. 6, in accordance with aspects of the present disclosure.

FIG. 8 shows a perspective view of a pin of the drive assembly of FIG. 6, in accordance with aspects of the present disclosure.

FIGS. 9A, 9B, and 9C show an end view, a first cross-sectional view, and a second cross-sectional view of a rack of the drive assembly of FIG. 6, in accordance with aspects of the present disclosure.

FIG. 10 shows a close-up perspective view of a distal end portion of the tissue removal device of FIG. 1, in accordance with aspects of the present disclosure.

FIG. 11 shows a perspective view of a holder of the distal end portion of FIG. 10, in accordance with aspects of the present disclosure.

FIGS. 12A and 12B show examples of end effectors, in accordance with aspects of the present disclosure.

FIGS. 13A, 13B, 14A, 14B, 15A, and 15B show an area of the tissue removal device of FIG. 1 in various operational states, in accordance with aspects of the present disclosure.

FIG. 16 shows a side view of a drive assembly for a tissue removal device, in accordance with aspects of the present disclosure.

FIGS. 17 and 18 show cross-sectional views of a tissue removal device in various operational states, the tissue removal device having the drive assembly of FIG. 16, in accordance with aspects of the present disclosure.

FIG. 19 shows a partially transparent view of a tissue removal device, in accordance with aspects of the present disclosure.

FIG. 20 shows an end view of a rotatable member of the tissue removal device of FIG. 19, in accordance with aspects of the present disclosure.

FIG. 21 shows a distal section of a drive member of the tissue removal device of FIG. 19, in accordance with aspects of the present disclosure.

FIG. 22 shows a cross-sectional view of distal end of a handle assembly of the tissue removal device of FIGS. 17 and 18, in accordance with aspects of the present disclosure.

### DETAILED DESCRIPTION

Reference now will be made in detail to aspects of the present disclosure, examples of which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. Though the following description refers to “ureteroscope” or “ureteroscopy,” the principles/aspects described herein may be used with any suitable introduction sheath or device, even if such sheath or device fails to include one or more features typically associated with “endoscopes.” It may be understood that both the foregoing general description and the following detailed description are exemplary and explanatory only and are not restrictive of the features claimed. Further, as used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” The terms “substantially,” “approximately” and “about” refer to a variation of plus or minus ten percent with respect to a stated value.

The present disclosure is drawn to tissue removal devices and related methods. On example of a tissue removal device is an ureteroscopic biopsy forceps that has opposing jaws. When the jaws are actuated by a user, the jaws may bite deep into a tissue mass and then tug a sample from the tissue mass with a single, smooth, continuous, or otherwise controlled action from the user, thereby helping to ensure that the jaws successfully harvest the sample. Additionally or alternatively, after the jaws are closed on the tissue mass, the user may rotate the jaws to sever the sample from the tissue mass. Optionally, an exemplary tissue removal device may include means for monopolar cauterizing of a harvest site to help ensure that any bleeding from the site is quickly resolved. The means may entail passing a low radiofrequency (RF) energy through the jaws at the harvest site to a receptor typically underneath the subject. This may produce a “burn” appearance at the harvest site. A more detailed discussion of these and other features can be found in the paragraphs below.

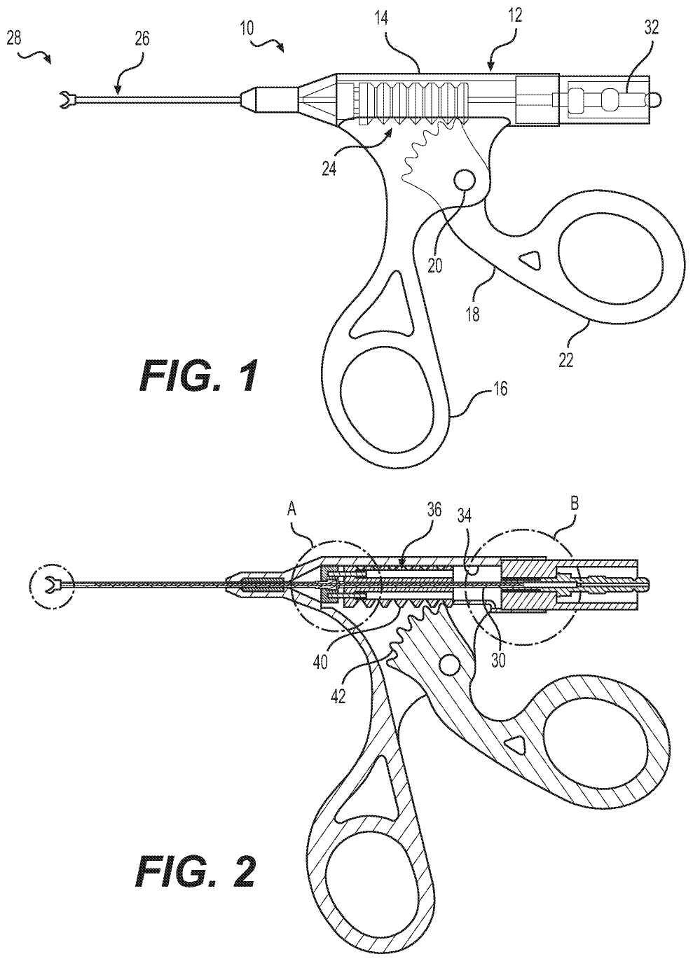

An exemplary tissue removal device 10 is shown in FIG. 1. Tissue removal device 10 may include a handle assembly 12. Handle assembly 12 may include multiple components for gripping by the user. For example, handle assembly may include a handle body 14 with a finger grip or loop 16 depending from its underside. Handle assembly 12 also may include a handle actuator 18 that may be movable relative to handle body 14. In one example, handle actuator 18 may rotate about a pivot 20 in clockwise and counterclockwise directions. Handle actuator 18 may include a finger grip 22. It is contemplated that a user may insert one or more fingers into finger grip 16 and a thumb into finger grip 22, and rotate handle actuator 18 relative to handle body 14 by bringing the fingers and thumb together and apart.

Tissue removal device 10 also may include a drive assembly 24, a shaft 26, an end effector 28 at a distal end of shaft 26, a drive member 30, and an electrical connector 32. Rotation of handle actuator 18 by the user may actuate drive assembly 24, leading to actuation of end effector 28 via drive member 30, and also to actuation of shaft 26, for obtaining a tissue sample from a tissue mass (not shown). Electrical connector 32 may receive electrical energy (e.g., low RF energy) from an external source (not shown), such as an electrosurgical generator. The electrical energy may flow through drive member 30 and then through end effector 28 to the tissue mass. The paragraphs below provide a more detailed description of theses and related components of tissue removal device 10.

Drive assembly 24 may include a longitudinally and/or linearly slidable member 36, shown in FIG. 2. In one example, member 36 may be a rack. Rack 36 may be located in a passage 34 in handle body 16. Rack 36 may be slidable in proximal and distal directions within passage 34. Rack 36 may be at least partially cylindrical. Rack 36 may include engagement elements, such as teeth 40, on its outer surface, with teeth 40 being spaced apart along the length of rack 36. Alternatively, rack 36 may include one or more slots or steps in place of teeth 40, or any other suitable engagement elements. Engagement elements, such as teeth 42 (or any other suitable engagement elements) on handle actuator 18, may allow handle actuator 18 to act as a rotatable pinion for driving linear movement of rack 36. For example, when handle actuator 18 rotates in the clockwise direction, teeth 42 may engage teeth 40 to slide rack 36 in a proximal direction within passage 34. On the other hand, where handle actuator 18 rotates in the counterclockwise direction, teeth 42 may engage teeth 40 to slide rack 36 in a distal direction within passage 34. Drive member 30 may be fixedly coupled to rack 36, such that drive member 30 may move proximally and distally together as a unit with rack 36. Drive member 30 may be operatively coupled to end effector 28, such that movement of drive member 30 may actuate end effector 28 to move between open and closed states.

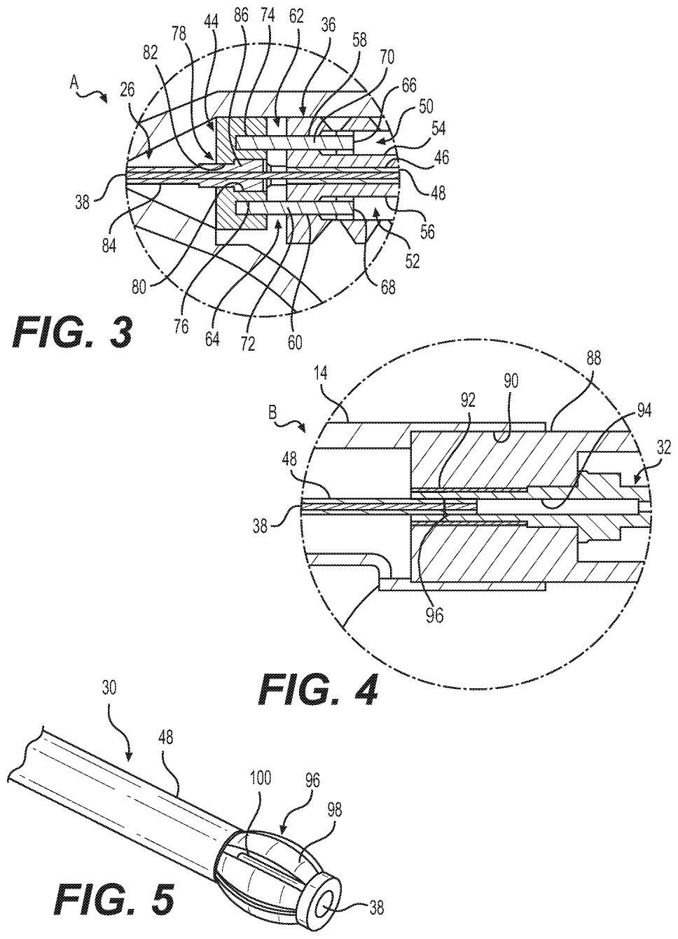

FIG. 3 is a close-up view of the area marked with an “A” in FIG. 2. A distal end portion of rack 36 is shown, along with a holder 44 also of drive assembly 24. Drive wire 38 is shown as being contained within a central passage 46 of rack 36. A sheath 48 around drive wire 38 may assist with fixing drive wire 38 to rack 36 within central passage 46. Drive wire 38 may extend distally out of rack 36. Rack 36 may include a plurality of offset passages 50 and 52 radially offset from central passage 46. Offset passages 50 and 52 may include proximal sections 54 and 56 and distal sections 58 and 60. The proximal sections 54 and 56 may be wider than the distal sections 58 and 60. Offset passages 50 and 52 may receive a plurality of pins 62 and 64, which may couple rack 36 to holder 44. While pins 62 and 64 may be movable relative to rack 36, pins 62 and 64 may be fixedly coupled to holder 44 such that pins 62 and 64 may move together as a unit with holder 44. Pins 62 and 64 may include heads 66 and 68 and stems 70 and 72. Heads 66 and 68 may be wider than stems 70 and 72. Stems 70 and 72 may fit in distal sections 58 and 60 of offset passages 50 and 52, such that stems 70 and 72 may be slidable through distal sections 58 and 60. Heads 66 and 68 may fit in proximal sections 54 and 56 of offset passages 50 and 52, such that heads 66 and 68 (and stems 70 and 72) may be slidable through proximal sections 54 and 56. Heads 66 and 68 may be too wide, however, to fit in distal sections 58 and 60. As such, pins 62 and 64 may allow rack 36 to move away from holder 44 for a range of motion corresponding to travel of heads 66 and 68 within proximal sections 54 and 56. Pins 62 and 64 may prevent rack 36 from moving away from holder 44 when heads 66 and 68 abut the wall surface (i.e., of the shoulder or step) where proximal sections 54 and 56 transition to distal sections 58 and 60.

In one example, the distal end portions of pins 62 and 64 may be received in offset passages 74 and 76 of holder 44. Offset passage 74 and 76 may be radially offset from a central passage 78 of holder. The distal end portions of pins 62 and 64 may be bonded, fused, or otherwise fixedly coupled to holder 44. Rack 36 may be slidable proximally and distally relative to holder 44 along pins 62 and 64. Accordingly, drive wire 38, which may be fixedly coupled to rack 36, may be slidable proximally and distally relative to holder 44. While two pins 62 and 64, and corresponding passages for the two pins 62 and 64, are shown and described in this disclosure, it should be understood that fewer or more pins and passages may be used.

In one example, drive wire 38 may extend through central passage 78 of holder 44. Central passage 78 may include a proximal section 80 and a distal section 82. Proximal section 80 may be wider than distal section 82. Passage 78 may receive a sheath 84. For example, sheath 84 may include an enlarged end 86 for receipt in central passage 78, to fixedly couple sheath 84 to holder 44, such that sheath 84 and holder 44 may move together as a unit. Accordingly, when rack 36 moves relative to holder 44, drive wire 38 may move relative to sheath 84. When rack 36 moves together in a unit with holder 44, drive wire 38 may move together as a unit with sheath 84. Sheath 84 may include, in one embodiment, a coil sheath. Sheath 84 may be electrically non-conductive. Additionally or alternatively, sheath 84 may be coated or covered by a layer of non-conductive material.

FIG. 4 is a close-up view of the area marked with a “B” in FIG. 2. A proximal end portion of device 10 is shown, where a proximal end portion of a handle insert 88 is received within an opening 90 in handle body 14. The proximal end portion of handle insert 88 may be fixedly coupled to handle body 14. For example, an outer surface of the proximal end portion of handle insert 88 may be fixedly coupled to the inner surface of handle body that defines opening 90 using adhesive, bonding, fusing, threaded engagement (e.g., screw threads), and/or any other suitable fixing means. Handle insert 88 may include a passage 92 extending longitudinally therethrough. Passage 92 may receive a distal end portion of electrical connector 32. The distal end portion of electrical connector 32 may be fixedly coupled to the portion of handle insert 88 defining passage 92 by any suitable fixing means, including those described above. In use, handle insert 88 and electrical connector 32 may be positionally fixed relative to handle body 14.

FIG. 5 is a close-up view of a proximal end portion of drive member 30. The proximal end portion of drive member 30 may include a proximal end portion of drive wire 38 and a proximal end portion of sheath 48 surrounding the proximal end portion of drive wire 38. The proximal end portion of drive member 30 may be slidably received within a passage 94 extending longitudinally through electrical connector 32. In embodiments of device 10 where electrical energy is delivered from electrical connector 32 to end effector 28, contact between the proximal end portion of drive member 30 and electrical connector 32 needs to be maintained. Because drive member 30 may move proximally and distally relative to handle body 14, or move in any other manner, while electrical connector 32 may remain stationary relative to handle body 14, fixedly coupling the proximal end portion of drive member 30 to electrical connector 32 may not be feasible. In one example, sheath 48 may include an enlarged or bulbous end 96. Enlarged end 96 may be formed by a plurality of bowed or convex portions 98 separated by a plurality of slits 100. The bowed portions 98 may be self-biased to move into the radially-outward extended positions shown in FIG. 5. In their extended positions, each of the bowed portions 98 may be spaced apart from an outer surface of drive wire 38. The bowed portions 98 may be compressed into radially-inward compressed positions shown in FIG. 4. In the radially-inwardly compressed positions, each of the bowed portions 98 may be pressed against the outer surface of drive wire 38, and/or slits 100 may be closed. The amount of expansion/compression of the bowed portions 98 may depend on the dimensions of passage 94. It is contemplated that the bowed portions 98 may be partially expanded/compressed when in passage 94, such that bowed portions 98 may compress inward to facilitate movement of drive member 30, and/or expand outward so as remain in contact with the surfaces defining passage 94, to accommodate whatever way drive member 30 moves relative to electrical connector 32 during use of device 10.

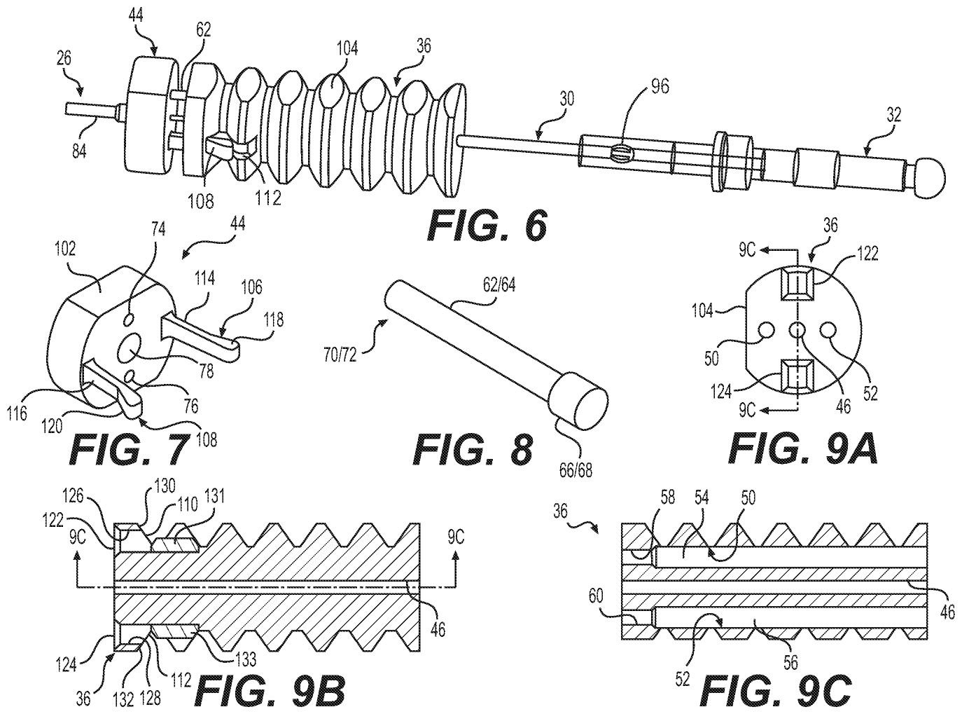

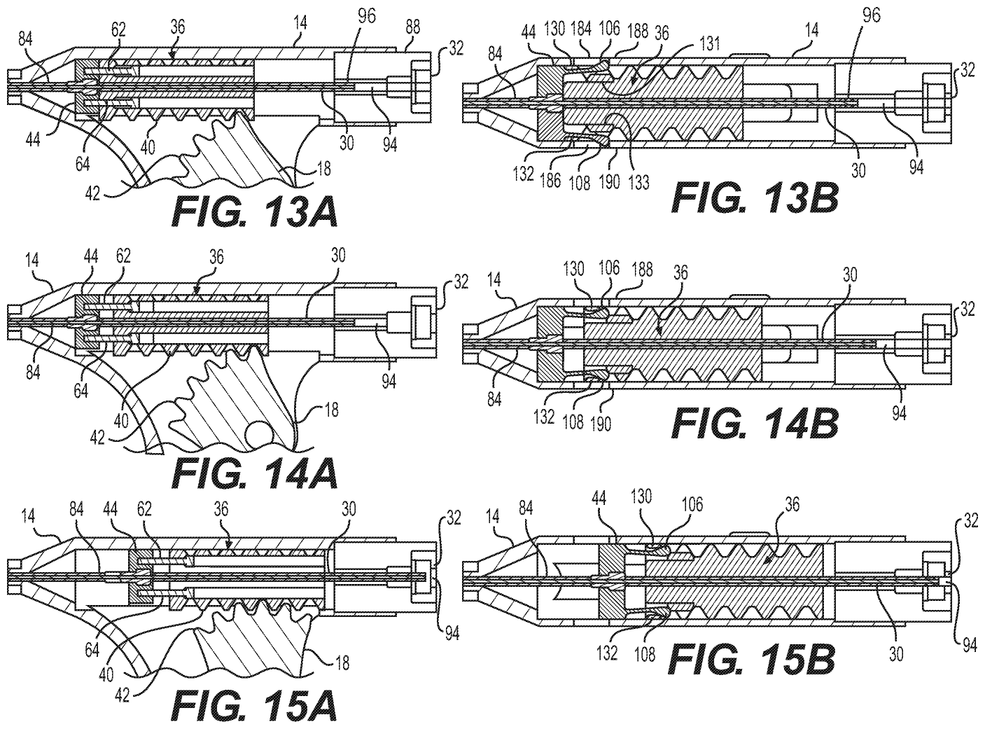

FIG. 6 shows, from left to right, shaft 26, holder 44, pins 62 and 64, rack 36, drive member 30, and electrical connector 32, assembled together. FIG. 7 shows holder 44 in isolation. Holder 44 may be substantially cylindrical. In one example, holder 44 may include a flattened region 102. It is contemplated that flattened region 102 may contact a flattened region (not shown) of handle body 14 that defines a portion of passage 34. The engagement between the flattened regions may inhibit or prevent holder 44 from rotating within passage 34, while still allowing holder 44 to translate proximally and distally within passage 34. Rack 36 also may include flattened regions 104 for similar purposes. By inhibiting or preventing holder 44 and rack 36 from rotating within passage 34, it can be ensured that holder 44 and rack 36 may be properly positioned relative to the rest of handle body 14. Proper positioning will be described in more detail when describing FIGS. 13A, 13B, 14A, 14B, 15A, and 15B below.

As shown in FIGS. 6 and 7, holder 44 may include one or more latching elements, such as proximally-extending tangs 106 and 108, that may be received by rack 36. Tangs 106 and 108 may include proximal stems 114 and 116. Tangs 106 and 108 also may include free end portions with angled surfaces 118 and 120. In FIG. 6, free end portions of tangs 106 and 108 are shown protruding from slots 110 and 112 in lateral surfaces of rack 36.

Tangs 106 and 108 may occupy the positions shown in FIGS. 6 and 7 in the absence of external forces exerted on tangs 106 and 108. Tangs 106 and 108 may be deformed by, for example, forcing tangs 106 and 108 toward each other, or by forcing tangs 106 and 108 away from each other. Tangs 106 and 108 may be elastically deformable such that they may move back to their rest positions when the deforming forces are removed. In one example, stems 114 and 116 of tangs 106 and 108 may bend under stress allowing the free end portions of tangs 106 and 108 to move radially inwardly or radially outwardly, and afterwards may straighten, with the free end portions of tangs 106 and 108 moving back to their rest positions when the stress is removed.

In one example, tangs 106 and 108 may be forced radially outwardly and radially inwardly by rack 36. Features of rack 36 are shown in the multiple views provided in FIGS. 9A-9C. Tangs 106 and 108 may extend proximally through openings 122 and 124 into passages 126 and 128 of rack 36. When rack 36 is moved distally into abutment with holder 44, shoulders 131 and 133 of rack 36 may force the free end portions of tangs 106 and 108 to deflect radially outwardly such that the free end portions protrude radially outwardly from passages 126 and 128 and slots 110 and 112 of rack 36. During use of device 10, as rack 36 moves proximally relative to holder 44 by sliding along pins 62 and 64 fixed to holder 44, shoulders 131 and 133 may be moved out of engagement with tangs 106 and 108, and shoulders 130 and 132 at the distal ends of slots 110 and 112 may move into engagement with tangs 106 and 108. For example, shoulders 130 and 132 may move into engagement with angled surfaces 118 and 120 of tangs 106 and 108. The engagement of shoulders 130 and 132 with angled surfaces 118 and 120 may force tangs 106 and 108 radially inwardly, and the deformation may increase as shoulders 130 and 132 continue to slide proximally over angled surfaces 118 and 120. Proximal movement of rack 36 relative to holder 44 may be arrested when heads 66 and 68 of pins 62 and 64 reach the distal ends of wide sections 54 and 56 of offset passages 50 and 52, and cannot enter the proximal ends of narrow sections 58 and 60. At that stage, holder 44 and rack 36 may move proximally together as a unit.

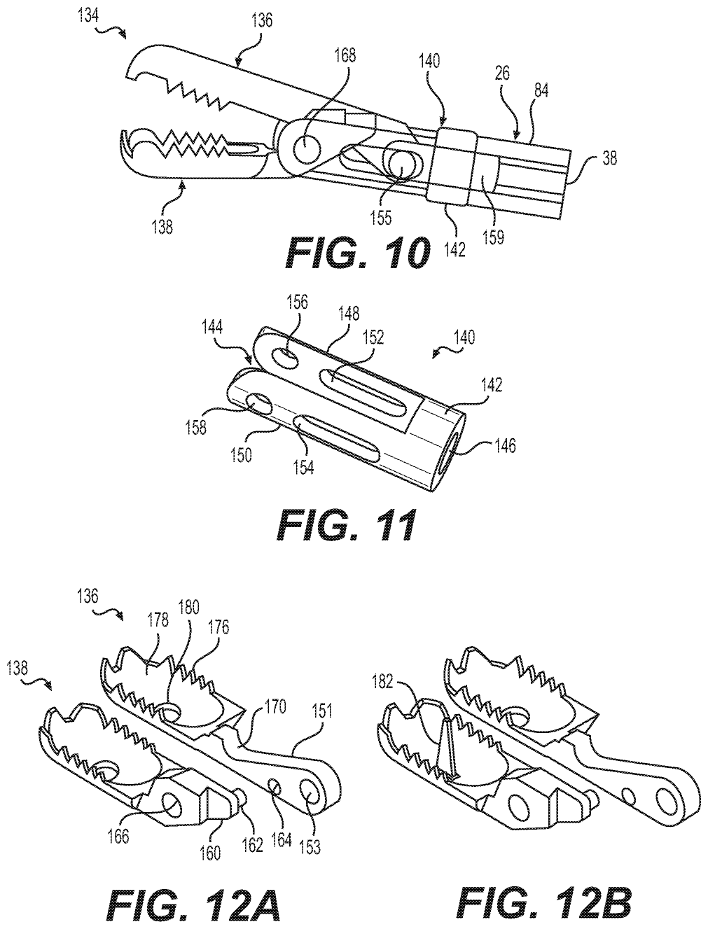

FIG. 10 shows an exemplary distal end portion of device 10. The distal end portion of device 10 may include any suitable end effector for obtaining a tissue sample, including forceps, a snare, scissors, a retrieval devices (e.g., a basket), and the like. It is contemplated that the snare and the basket may be self-expanding. FIGS. 10, 11, 12A, and 12B focus primarily on the use of forceps as the end effector. Forceps 134 may include jaws 136 and 138 that may move between an open state (FIG. 1) and a closed state (not shown). In a fully closed state, one or more surfaces of jaw 136 may contact jaw 138.

Jaws 136 and 138 may be movably coupled to a holder 140. Holder 140 may include a base 142. Base 142 may include a passage 146 extending therethrough. Holder 140 may be fixedly coupled to sheath 84 at a proximal end of base 142. A coupler 159, fixedly coupled to drive wire 38, may slide proximally and distally through passage 146. Arms 148 and 150 of holder 140 may extend distally from base 142. A cavity 144 may be formed between arms 148 and 150 and base 142. Portions of jaws 136 and 138 may be received by cavity 144. Arms 148 and 150 may include slots 152 and 154 and holes 156 and 158 distal to slots 152 and 154. Slots 152 and 154 and holes 156 and 158 may receive pins or rivets 155 and 168, respectively. Pin 155 may rotatably couple a proximal end portion 151 of jaw 136 to coupler 159. Pin 155 may slide proximally and distally within slots 152 and 153. Pin 168 may rotatably couple a proximal end portion 160 of jaw 138 to holder 140. Pin 168 may extend through hole 166 and holes 156 and 158. Jaw 136 may include a cutout 170 configured to receive pin 168, so as not to obstruct the passage of pin 168 through holes 156 and 158, while also allowing jaw 136 to be moved away from pin 168.

As shown in FIG. 12A, proximal end portion 151 of jaw 136 may include a hole 153 extending sideways therethrough. Hole 153 may receive pin 155. Proximal end portion 151 may be moved proximally and distally by proximal and distal movement of drive wire 38, via pin 155. Proximal end portion 151 also may be rotated in clockwise and counterclockwise directions about pin 155, due to movement of drive wire in distal and proximal directions.

Proximal end portion 160 of jaw 138 may include a pin 162. Pin 162 may be received in a hole 164 in jaw 136. Pin 162 may rotatably couple jaws 136 and 138. Jaw 138 also may include a hole 166 for receiving pin 168. Jaw 138 may be moved in clockwise and counterclockwise directions about pin 168 by movement of proximal end portion 151 of jaw 136 in proximal and distal directions, via rotation of pin 162 of jaw 138 in hole 164 of jaw 136.

Distal portion 172 of jaw 136 may be serrated. For example, distal portion 172 may include teeth 176 surrounding a recess 178. Recess 178 may receive the tissue sample, while teeth 176 may help with gripping onto the tissue sample. Jaw 136 may include an aperture 180 in recess 178. Distal portions 172 and 174 of jaws 136 and 138 may be similar. The opposing teeth of distal portions 172 and 174 may be complementary, such that their teeth may interlock to allow jaws 136 and 138 to fully close. As shown in FIG. 12B, the aperture of at least one of jaws 136 and 138 may be replaced with a protrusion, fin, or pin 182. Protrusion 182 may firmly engage with the tissue specimen (e.g., by piercing the tissue specimen) to facilitate its removal from the tissue mass using jaws 136 and 138.

FIGS. 13A, 13B, 14A, 14B, 15A, and 15B show device 10 in various stages of operation. With respect to the figure number for these figures, the “A” designation corresponds to a side view, while the “B” designation corresponds to a top view. In FIGS. 13A and 13B, handle actuator 18 has been rotated in the counterclockwise direction so as to move rack 36 and holder 44 into their distal-most positions. Further movement of rack 36 and holder 44 in the distal direction may be hindered by holder 44 abutting against a distal tapered section of handle body 14 (see, e.g., FIGS. 17 and 18). The distal end face of rack 36 may abut against holder 44 when both are in their distal-most positions.

As shown in FIG. 13A, heads 66 and 68 of pins 62 and 64 may be in their proximal-most positions in offset passages 50 and 52 of rack 36. Although not shown, end effector 28 (e.g., forceps 134) may be in an open or otherwise expanded position in preparation for receiving a portion of the tissue mass, due to the distal movement of drive wire 38 forcing coupler 159, pin 155, and jaw 136 distally along slots 152 and 154 of holder 140 (FIG. 10).

As shown in FIG. 13B, tangs 106 and 108 are their radially-outward deflected states due to the engagement of shoulders 131 and 133 with tangs 106 and 108. As such, the free end portions of tangs 106 and 108 may protrude outwardly from slots 110 and 112 of rack 36. The free end portions of tangs 106 and 108 may project into slots 184 and 186 in handle body 14. The tips of tangs 106 and 108 may be aligned with shoulders 188 and 190 formed by proximal ends of slots 184 and 186. Abutting engagement between the tips of tangs 106 and 108 and shoulders 188 and 190 may prevent holder 44 from being moved in the proximal direction.

The user may rotate handle actuator 18 in the clockwise direction to move device 10 from the state shown in FIGS. 13A and 13B to the state shown in FIGS. 14A and 14B. As handle actuator 18 rotates, it may draw rack 36 in the proximal direction. As rack 36 begins moving in the proximal direction, holder 44 may remain stationary. This is due to the engagement between the tips of tangs 106 and 108 with shoulders 188 and 190. Rack 36 may slide away from holder 44 along pins 62 and 64. As rack 36 slides away from holder 44, rack 36, which may be fixedly coupled to drive wire 38, may draw drive wire 38 proximally. Sheath 84, which may be fixedly coupled to holder 44, may remain stationary. As drive wire 38 moves proximally relative to sheath 84, coupler 159, pin 155, and jaw 136 may move proximally relative to holder 140, pin 168, and jaw 138 (FIG. 10), resulting in jaws 136 and 138 moving toward each other toward their fully-closed state. This is when jaws 136 and 138 bite the tissue specimen.

Continued clockwise rotation of handle actuator 18 may continue to move rack 36 proximally until heads 66 and 68 of pins 62 and 64 reach the distal ends of the wide sections 54 and 56 of offset passages 52 and 56 of rack 36. During this movement, shoulders 131 and 133 may move out of engagement with tangs 106 and 108, and shoulders 130 and 132 may move into engagement with tangs 106 and 108. As shoulders 130 and 132 ride proximally along angled surfaces 118 and 120 of the free end portions of tangs 106 and 108, shoulders 130 and 132 may deflect tangs 106 and 108 radially inwardly, as shown in FIG. 14B. This may move the tips of tangs 106 and 108 out of slots 184 and 186 of handle body 14. At this stage, jaws 136 and 138 may be in their fully-closed states, or at least as close to their fully-closed states as possible with the tissue specimen being grasped therein.

Further clockwise rotation of handle actuator 18 may move rack 36 and holder 44 proximally together as a unit, due to engagement between heads 66 and 68 of pins 62 and 64 with the step or shoulder in offset passages 50 and 52 of rack 36, and clearance of tangs 106 and 108 from slots 184 and 186 of handle body 14. This is exemplified by the change of device 10 from the state shown in FIGS. 14A and 14B to the state shown in FIGS. 15A and 15B. In FIGS. 15A and 15B, rack 36 and holder 44 have already been moved together as a unit for a distance in the proximal direction. As such, with jaws 136 and 138 remaining in their closed positions for grasping the tissue specimen, jaws 136 and 138 and shaft 126 may move together as a unit in the proximal direction. Sheath 84, which may be fixedly coupled to holder 44, and drive wire 38, which may be fixedly coupled to rack 36, may move in unison as rack 36 draws holder 44 at the urging of handle actuator 18. These movements result in jaws 136 and 138 tugging, jerking, tearing, or otherwise separating the tissue specimen away from the tissue mass. The tissue specimen may then be removed from the subject for analysis. Additionally or alternatively, electrical energy may be supplied to jaws 136 and 138, and introduced into the sample site, to stem or otherwise reduce bleeding at the site.

The tug may be performed without the user having to move handle assembly 12 proximally with a quick, jerking action. The tug described herein, due to it being generated by rotation of handle actuator 18 rather than the user manipulating a larger portion or all of device 10, may be more consistent and repeatable than conventional methods, which may reduce the likelihood of operator error. Additionally, because a continuous or otherwise smooth rotation of handle actuator 18 results in grasping of the tissue specimen and tugging the tissue specimen away, device 10 may be easier to use overall than conventional devices.

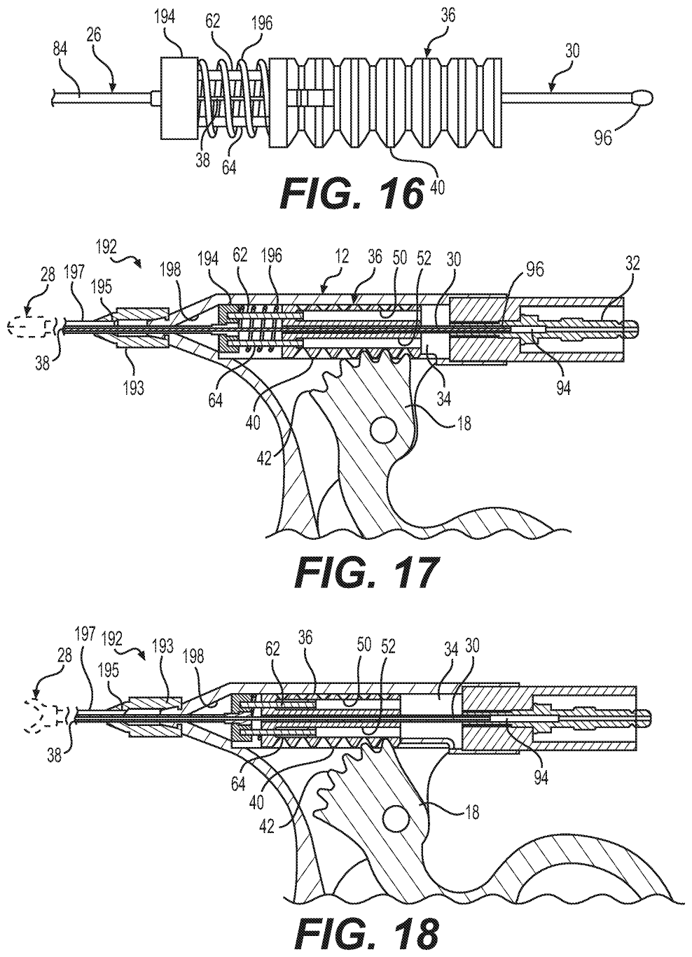

FIGS. 16-18 show another exemplary tissue removal device 192. Device 192 may include many of the same components as device 10. Parts in devices 10 and 192 that are the same or similar have been assigned the same reference numbers.

FIG. 16 shows a portion of the internals of device 192, including shaft 26 (sheath 84 and drive member 38), pins 62 and 64, rack 36, and drive member 30. These components may form a core of a drive assembly of device 192. One difference between device 192 and device 10 is that device 192 has a holder 194 that is devoid of tangs 106 and 108. Additionally, a biasing member 196 extends from a proximal end face of holder 194 to a distal end face of rack 36. Biasing member 196 urges holder 194 away from rack 36, thereby creating a gap between the proximal end face of holder 194 and the distal end face of rack 36. The size of the gap is dependent on the length of pins 62 and 64. The spaced positioning of holder 194 relative to rack 36 corresponds to end effector 28 being in its closed position, similar to the situation depicted in FIGS. 14A and 14B for device 10. Thus, device 192 has a normally-closed state depicted in FIG. 16.

FIG. 17 shows the drive assembly of FIG. 16 within handle assembly 12. Biasing member 196 may position holder 194 in its distal-most position up against a tapered section 198 of handle body 14. Biasing member 196 also may position rack 36 a predetermined distance from holder 194. Heads 66 and 68 of pins 62 and 64 may be at their distal-most positions within wide sections 54 and 56 of offset passage 50 and 52 of rack 36, thereby limiting rack 36 from moving any farther away from holder 194.

Moving device 192 from the state of FIG. 17 to the state of FIG. 18 may be carried out by rotating handle actuator 18 in the counterclockwise direction. The counterclockwise rotation may move rack 36 in the distal direction, due to the engagement between teeth 42 of handle actuator 18 and teeth 40 of rack 36. The distal movement of rack 36, coupled with holder 194 remaining stationary due to its engagement with tapered section 198, may result in drive wire 38 moving distally relative to sheath 84. Drive wire 38 moving distally relative to sheath 84 may result in end effector 28 (e.g., forceps 134) moving toward its open position (see FIG. 10). The distal movement of rack 36 relative to holder 194 also may compress biasing member 196. Rack 36 may slide along pins 62 and 64 in the proximal direction.

The user may position the open jaws 136 and 138 on an area of a tissue mass, and may release handle actuator 18. Biasing member 196 may expand, thereby moving rack 36 away from holder 194 and back toward the position shown in FIG. 17. Accordingly, jaws 136 and 138 may close onto the area of the tissue mass with a closing force corresponding to the restoring force of biasing member 196 expanding from its compressed state. As such, the closing force may be exerted consistently and repeatedly without error that could be introduced by closing jaws 136 and 138 by manual actuation. Biasing member 196 also may rotate handle actuator 18 clockwise when going from the state shown in FIG. 18 back to the state shown in FIG. 17.

From the state shown in FIG. 17, further clockwise rotation of handle actuator 18 by the user may move rack 36 and holder 194 together as a unit in the proximal direction, due to their connection via pins 62 and 64. Biasing member 196 may continue to keep holder 194 and rack 36 spaced apart from each other. As such, jaws 136 and 138, and shaft 26, may be pulled together in the proximal direction, with jaws 136 and 138 remaining closed, to tug, jerk, tear, or otherwise separate the tissue specimen from the tissue mass. The tissue specimen may be withdrawn from the subject, and then release using steps similar to those described in the transition between the state of FIG. 17 to the state of FIG. 18.

Additionally or alternatively, device 192 may include a rotatable cap 193. Rotatable cap 193 may be rotatably mounted to a distal-most end of handle body 14. As shown in FIG. 22, rotatable cap 193 may include a keyhole passage 195 extending therethrough. One example of a keyhole passage is a passage with a cylindrical portion and a slot portion extending outwardly from the cylindrical portion. Keyhole passage 195 may receive sheath 84 and a protrusion 197 (e.g., a spine, ridge, or the like) protruding radially outwardly from a surface of sheath 84. Due to the engagement between protrusion 197 and surfaces of keyhole passage 195, rotation of rotatable cap 193 may rotate sheath 84, and thus, may rotate holder 140, pins 155 and 168, and jaws 136 and 138. This rotation may allow jaws 136 and 138 to twist a tissue specimen to help separate the tissue specimen from the larger tissue mass. These rotational features may be used with any of the other aforementioned embodiments. Drive wire 38 may be rotatably coupled to coupler 159 to facilitate rotation of forceps 134. Additionally or alternatively, sheath 84 may be rotatably coupled to, but positionally fixed (at least longitudinally) relative to holder 194 to facilitate rotation of sheath 84 and forceps 134.

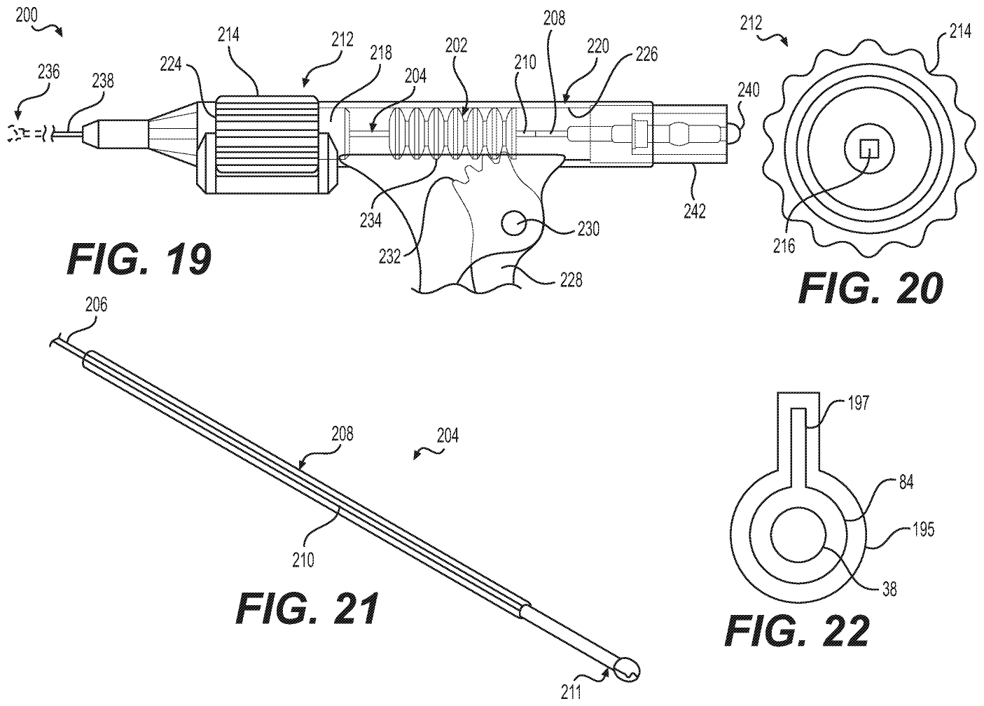

FIG. 19 shows another example of a tissue removing device 200. Tissue removing device 200 may include a rack 202, a drive member 204, a drive wire 206, a sheath 208, a handle body 220, a passage 226, a handle actuator 228, a pivot pin 230, teeth 232, teeth 234, an end effector 236, a sheath 238, an electrical connector 240, and a handle insert 242, similar, for example, to corresponding features of devices 10 and 192.

One difference between device 200 and devices 10 and 192 is the absence of longitudinally slidable holders 44 and 194. Device 200 may include a rotatable holder 212 fixedly coupled to sheath 238. End effector 236 may be at the distal end of sheath 238. End effector 236 may be similar to end effector 28, and sheath 238 may be similar to sheath 84. Rotatable holder 212 may be received in an opening 224 in handle body 220. One or more reduced-width end portions of rotatable holder 212, and/or any other suitable rotational support elements, may be received in passage 226 to rotatably mount rotatable holder 212 to handle body 220. A manipulation portion 213 of rotatable holder 212 may be exposed from opening 224, allowing the user to manipulate portion 213 so as to rotate rotatable holder 212. Because sheath 238 is fixedly coupled to rotatable holder 212, and end effector 236 may be coupled to sheath 238, rotation of sheath 238 may rotate end effector 236.

Another difference between device 200 and devices 10 and 192 is that rack 202 may be rotatable within passage 226. In one example, drive member 204 is fixedly coupled to rack 202. Drive member 204 may include a polygonal portion 210 (FIG. 21). Polygonal portion 210 may be formed by an additional sheath 208 applied to the outside of drive member 204, or may be formed of the material of drive member 204 itself. Polygonal portion 210 may be received in a polygonal passage 216 (FIG. 20) in rotatable holder 212. Due to the complementary profiles of polygonal portion 210 and polygonal passage 216, rotation of rotatable holder 212 by the user may impart rotation to drive member 204, and rack 202. This may also result in rotation of drive wire 206 and end effector 236, due to coupling between end effector 236 and drive wire 206. The rotation of end effector 236 may help twist or otherwise separate the tissue specimen from the tissue mass. End effector rotation may be performed in combination with the closing of end effector 236 on the tissue specimen, which may be driven by rotation of handle actuator 228 moving rack 202 distally (and thus, drive member 204 and drive wire 206). Drive member 204 may include an enlarged or bulbous end 211, similar to enlarged end 96, so that engagement between drive member 204 and the insides of electrical connector 240 may be maintained during rotational and/or translation movement of drive member 204. Electrical energy may be introduced from an outside source and conducted through electrical connector 240, through drive member 204, into drive wire 206, to end effector 236, and into the sample site.

It will be apparent to those skilled in the art that various modifications and variations can be made in the disclosed devices and related methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A tissue removal device, comprising:
a handle assembly;
a sheath coupled to the handle assembly, wherein a first portion of the sheath is within the handle assembly and a second portion of the sheath protrudes from the handle assembly, and wherein the sheath includes a passage extending therethrough;
an end effector coupled to an end of the sheath; and
a drive member coupled to the handle assembly and to the end effector, wherein the drive member extends through the passage of the sheath to the end effector,
wherein actuation of the handle assembly moves the end effector between an open state, for receiving material, and a closed state, for holding onto material, by causing relative movement between the drive member and the sheath, and
wherein further actuation of the handle assembly moves the end effector relative to the handle assembly while the end effector remains in the closed state.

2. The tissue removal device of claim 1, wherein the handle assembly includes a handle body and a handle actuator, wherein the handle actuator is movable relative to the handle body, and wherein actuation of the handle assembly includes moving the handle actuator relative to the handle body.

3. The tissue removal device of claim 2, wherein the further actuation of the handle assembly draws the end effector toward the handle body while the end effector remains in the closed state.

4. The tissue removal device of claim 2, wherein the further actuation of the handle assembly draws the sheath toward the handle body while the end effector remains in the closed state.

5. The tissue removal device of claim 2, wherein the handle assembly includes:
a first member movably mounted on the handle body, wherein the first member is fixedly coupled to the drive member,
a second member movably mounted on the handle body, wherein the second member is fixedly coupled to the sheath, and wherein the first member is movably coupled to the second member.

6. The tissue removal device of claim 5, wherein the second member includes one or more latching elements, wherein in a first state of the one or more latching elements, the one or more latching elements latch the second member to the handle body, such that the second member is positionally fixed on the handle body, and wherein in a second state of the one or more latching elements, the one or more latching elements release the second member from the handle body, such that the second member is movable relative to the handle body.

7. The tissue removal device of claim 6, wherein movement of the first member moves the one or more latching elements to the first state.

8. The tissue removal device of claim 6, wherein movement of the first member moves the one or more latching elements to the second state.

9. The tissue removal device of claim 5, wherein a biasing member has a first end engaging the first member and a second end engaging the second member.

10. The tissue removal device of claim 9, wherein the biasing member is configured to bias at least one of the first member and the second member toward a configuration in which the first member is a predetermined distance from the second member, to move the end effector toward the closed state.

11. The tissue removal device of claim 1, wherein the further actuation of the handle assembly moves the end effector relative to the handle assembly by translationally moving the end effector along an axis coaxial with a longitudinal axis of the sheath.

12. The tissue removal device of claim 1, wherein the further actuation of the handle assembly moves the end effector relative to the handle assembly by rotating the end effector about an axis coaxial with a longitudinal axis of the sheath.

13. A tissue removal device, comprising:
a handle assembly supporting a first member and a second member;
a sheath, wherein a proximal portion of the sheath extends into the handle assembly, and a distal portion of the sheath protrudes from the handle assembly, wherein the sheath includes a passage extending therethrough, and wherein a proximal end of the sheath is fixedly coupled to the first member;
an end effector coupled to a distal end of the sheath; and
a drive member, wherein the drive member extends through the passage of the sheath, and wherein a distal end of the drive member is coupled to the end effector, and a proximal end of the drive member is fixedly coupled to the second member, wherein the first member and the second member are coupled so as to be movable relative to each other in a first state of the tissue removal device, and to move together as a unit in a second state of the tissue removal device.

14. The tissue removal device of claim 13, wherein the first member and the second member are coupled by one or more pins, wherein the second member is movable away from the first member by sliding along the one or more pins when the tissue removal device is in the first state, and wherein the second member is prevented from moving away from the first member by the one or more pins when the tissue removal device is in the second state.

15. The tissue removal device of claim 13, further including a compression spring engaging opposing surfaces of the first member and the second member.

16. The tissue removal device of claim 13, wherein the first member includes one or more latching elements, and wherein the one or more latching elements are movable between a latching state, in which the one or more latching elements latch the first member to the handle assembly, thereby positionally fixing the first member relative to the handle assembly, and a releasing state, in which the one or more latching elements unlatch from the handle assembly, thereby allowing the first member to move relative to the handle assembly.

17. The tissue removal device of claim 16, wherein movement of the second member relative to the first member causes the second member to move the one or more latching elements between the latching state and the releasing state.

18. A tissue removal method that uses a tissue removal device to remove a tissue specimen from a tissue mass of a subject, the method comprising:
holding a handle assembly of the tissue removal device;
guiding a shaft of the tissue removal device and an end effector of the tissue removal device toward the tissue mass to position the end effector proximate a target area of the tissue mass, wherein the shaft protrudes from the handle assembly, and wherein the shaft includes a sheath having an end supporting the end effector, and wherein the shaft further includes a drive member extending through the sheath, the drive member being coupled to the end effector;
actuating the handle assembly to move the end effector between an open state for receiving a tissue specimen from the target area and a closed state for holding onto the target area, wherein movement of the end effector between states is at least partially driven by movement of the drive member relative to the sheath; and
further actuating the handle assembly to move the end effector relative to the handle assembly while the end effector remains in the closed state to pull the target area away from the rest of the tissue mass.

19. The tissue removal method of claim 18, wherein at least one of actuating the handle assembly and further actuating the handle assembly includes rotating the end effector about an axis coaxial with a longitudinal axis of the sheath.

20. The tissue removal method of claim 18, wherein further actuating the handle assembly to move the end effector relative to the handle assembly includes translationally moving the end effector along an axis coaxial with a longitudinal axis of the sheath.
